(19) United States
(12) Patent Application Publication
(10) Pub. No.: US 2024/0164717 A1
(43) Pub. Date: May 23, 2024

(54) THREE-DIMENSIONAL PRINTED ARTICLES AND METHODS OF MANUFACTURE

(71) Applicant: ARIZONA BOARD OF REGENTS ON BEHALF OF THE UNIVERSITY OF ARIZONA, Tucson, AZ (US)

(72) Inventors: TUCKER STUART, Tucson, AZ (US); PHILIPP GUTRUF, Tucson, AZ (US)

(21) Appl. No.: 18/281,527

(22) PCT Filed: Mar. 11, 2022
(86) PCT No.: PCT/US22/20030

§ 371 Date: Sep. 11, 2023

Related U.S. Application Data
(60) Provisional application No. 63/159,884, filed on Mar. 11, 2021.

(51) Int. Cl. A61B 5/00; B33Y 80/00; B81B 7/00

(52) U.S. Cl. CPC A61B 5/6831; B33Y 80/00; B81B 7/0061; B81B 2201/058

(57) ABSTRACT

Three-dimensionally (3D) printed, wearable, electronic sensing articles and methods of manufacture are disclosed.

### CROSS-REFERENCE TO RELATED APPLICATIONS

This application claims the benefit of U.S. provisional application No. 63/159,884 filed Mar. 11, 2021 which is incorporated by reference in its entirety.

### SUMMARY

Three-dimensionally (3D) printed, wearable, electronic sensing articles and methods of manufacture are disclosed. A 3D digital (computer) model of a body of a particular (unique) wearer may be obtained by 3D mapping, such as CT scan, MRI scan, photogrammetric mapping and/or 3D scanning, etc. Data from a 3D dataset of the 3D digital mathematical representation of the wearer's body may then be manipulated via software to create a two-dimensional (2D) template, particularly by unfolding the 3D dataset. The two-dimensional (2D) template may then be used to generate the 3D printed personalized wearable electronic article, which may comprise a mesh as a base structure, which is custom (uniquely) fitted to the wearer's body. The mesh may be formed of an elastomeric polymer composition, to provide a mesh which is flexible, soft and stretchable (elastic), whereby the mesh is conformable (e.g. circumferentially) to changes in body shape of the wearer's body during movement, particularly by elastic deformation. This individually tailored fit may provide an at least substantially perfect conformality to the wearer's body, enabling high-fidelity and chronic biodata acquisition that is adhesive-free (i.e. no adhesive is required to bond the sensing article to the body of the wearer). It may be understood that wearables that require use of an adhesive to be attached to the wearer's body generally may only be used for periods of continuous attachment which is less than one week, and which are at best limited to two weeks. However, given the wearable articles of the present disclosure do not require use of an adhesive to be attached to the wearer's body, the wearable articles of the present disclosure are not so limited and may be continuously attached to the wearer's body for periods which may exceed two weeks. Moreover, the wearable articles of the present disclosure may be removed from the body and subsequently reapplied, whereas wearables that require use of an adhesive to be attached to the wearer's body are generally single use.

### BACKGROUND

Current sensing technologies have recently advanced significantly with the introduction of epidermal electronics, epifluidics and wireless, battery-free electronics that enable recording and capture of clinical-grade biophysical data from subjects without need of cumbersome conventional tools that inhibit the wearer's mobility with a need for constant user or patient interaction. A common issue in the creation of these types of physiological, as well as biofluid, sensors systems is the complex development process and subsequently long development time that make it nearly impossible to create custom-made devices specifically tailored towards specific biomarkers or use cases. The advantage of a custom-made sensor system, however, would ensure the highest sensing fidelity through intimate skin contact in a form factor that does not require skin adhesives and reduces bulk to the point of imperceptible feel. Additional challenges include the user interaction that is required with current wearable systems, which either require the need to recharge batteries or wired hardware in close proximity of data collection devices, limiting experimental, clinical and commercial paradigms, inhibiting functional use cases significantly.

### BRIEF DESCRIPTION OF THE DRAWINGS

Features and advantages of this disclosure will become more apparent as the following detailed description proceeds, and upon reference to the drawings, wherein like numerals designate like parts, and in which:

FIG. 1 is a view of a system according to the present disclosure, particularly a healthcare monitoring and/or diagnostic system, which comprises a three-dimensionally (3D) printed sensing article;

FIG. 2 is a view of a three-dimensionally printed sensing article disposed on a human body;

FIG. 3 is a close-up view of the region of FIG. 2 bounded by rectangle 3;

FIG. 4 is a view of a three-dimensionally printed sensing article disposed on a human body, including an electronic structure, particularly an antenna structure for power receiving (harvesting) from an external wireless power source, and a close up cross-sectional view of electronic components of the electronic structure embedded/encapsulated in a three-dimensionally printed electronic enclosure of the electronic structure;

FIG. 5 is a close-up exploded view of an electronic structure of a three-dimensionally printed sensing article;

FIG. 6 is a close-up exploded view of an electronic structure of a three-dimensionally printed sensing article;

FIG. 6A is a close-up side view of a lower portion of an electronic enclosure of the electronic structure of FIG. 6;

FIG. 6B is a close-up perspective view of the lower portion of the electronic enclosure of the electronic structure of FIG. 6;

FIG. 6C are cross-sectional views of electronic component encapsulation in the electronic enclosure during fabrication of the three-dimensionally printed sensing article;

FIG. 7 is a graph of magnitude (decibels) as a function of frequency (gigahertz) of the antenna structure for varying distance/height (millimeters) of the antenna structure (e.g. from the body);

FIG. 8 a view of a system according to the present disclosure, particularly a healthcare monitoring and/or diagnostic system, which comprises a three-dimensionally printed sensing article comprising electronic components, such as wireless power receiving (harvesting) components, wireless communication components and electronic data collection (sensing) components;

FIG. 9 is a block diagram of a system according to the present disclosure, particularly a healthcare monitoring and/or diagnostic system, which comprises a three-dimensionally printed sensing article comprising electronic components, such as wireless power receiving (harvesting) components, wireless communication components and electronic data collection (sensing) components;

FIG. 10 is a graph of power (milliwatts) as a function of distance (meters) between the power castor/transmitting device and an antenna of a sensing article;

FIG. 11 is a sensing modality/component location map for electronic data collection (sensing) components of one or more three-dimensionally printed sensing articles;

FIG. 12 is a graph of temperature (degrees Celsius) as a function of time (minutes) for sensitivity and temporal resolution of an electronic data collection (sensing) component comprising a high-fidelity temperature sensor;

FIG. 13 is a graph of temperature (degrees Celsius) as a function of time (hours) for the electronic data collection (sensing) component comprising the high-fidelity temperature sensor showing circadian rhythm;

FIG. 14 is a graph of skin humidity (%, percent) as a function of time (minutes) for an electronic data collection (sensing) component comprising a skin humidity sensor;

FIG. 15 is a graph of acceleration (g-force) as a function of time (sec) for an electronic data collection (sensing) component comprising an accelerometer;

FIG. 16A is a sequence diagram showing three-dimensional digital data mapping of the surface of an appendage of a human body, particularly by photogrammetric mapping, to create a three-dimensional model of the surface of the appendage of the body;

FIG. 16B is a sequence diagram showing extraction of two-dimensional physiological data of a surface of the appendage of the body from the three-dimensional data/model;

FIG. 17A is a top view of a three-dimensionally printed sensing article, including a microfluidic structure, particularly including a microfluidic channel (for eccrine sweat collection) in a microfluidic channel enclosure, disposed in three-dimensional form on the body, particularly a forearm and extending around the proximal-distal axis;

FIG. 17B is a side view of the three-dimensionally printed microfluidic channel structure of the sensing article of FIG. 17A showing collection and release of sweat;

FIG. 17C is a top view of the of the three-dimensionally printed microfluidic structure, including the microfluidic channel in the microfluidic channel enclosure, of the sensing article of FIG. 17A;

FIG. 17D is a cross-sectional view of the three-dimensionally printed microfluidic structure, including the microfluidic channel in the microfluidic channel enclosure, of FIG. 17C taken along section line 17D-17D;

FIG. 17E is a cross-sectional view of the three-dimensionally printed microfluidic structure, including the microfluidic channel in the microfluidic channel enclosure, of FIG. 17C taken along section line 17E-17E;

FIG. 17F is cross-sectional (photographic) view of the three-dimensionally printed microfluidic structure, including the microfluidic channel in the microfluidic channel enclosure, of FIG. 17C taken along section line 17F-17F;

FIG. 17G is an exploded view of the microfluidic structure of the sensing article;

FIG. 17H is a sequence diagram showing sweat collection from a human body within a three-dimensionally printed microfluidic channel at 10 minutes intervals of 30 minute time period, particularly during callisthenic exercise;

FIG. 17I is a graph of total sweat volume (microliters) collected as a function of time (minutes) for the 30 minute time period;

FIG. 17J is a view of contact angle of water disposed on smooth and rough surfaces of three-dimensionally printed thermoplastic polyurethane for the sensing article;

FIG. 17K is a bar graph of contact angle of water disposed on smooth and rough surfaces of three-dimensionally printed thermoplastic polyurethane for the sensing article;

FIG. 17L is a graph of inlet pressure (kilopascals) to induce fluid (e.g. sweat) flow therethrough as a function of microfluidic channel area (square millimeters);

FIG. 18A is a view of an exemplary three-dimensionally printed sensing article including an electronic structure, particularly including a strain gauge structure comprising a strain gauge sensor embedded/encapsulated in a three-dimensionally printed strain gauge enclosure of the sensing article;

FIG. 18B is a view of the strain gauge structure of the three-dimensionally printed sensing article disposed on a body with other components;

FIG. 18C are cross-sectional views of the strain gauge sensor in the strain gauge enclosure during fabrication of the sensing article;

FIG. 19A is a view of a mesh structure of the sensing article in response to strains of 10%, 20% and 30% in the longitudinal direction;

FIG. 19B is a cross-sectional view of a strut of the mesh structure;

FIG. 19C is another cross-sectional view of a strut of the mesh structure;

FIG. 19D is a graph of stress versus strain curves displaying results of height and thickness modulation for the struts;

FIG. 20 is a view of a mesh structure for strain isolation of electronics; and

FIG. 21 is a view of an enclosure disposed on top of the struts of the mesh structure.

### DETAILED DESCRIPTION

It may be appreciated that the present disclosure is not limited in its application to the details of construction and the arrangement of components set forth in the following description or illustrated in the drawings. The invention(s) herein may be capable of other embodiments and of being practiced or being carried out in various ways. Also, it may be appreciated that the phraseology and terminology used herein is for the purpose of description and should not be regarded as limiting as such may be understood by one of skill in the art.

Throughout the description, like reference numerals and letters indicate corresponding structure throughout the drawings/embodiments and, as such, corresponding structure may not be separately discussed as merely being repetitive. Furthermore, any particular feature(s) of a particular exemplary embodiment may be equally applied to any other exemplary embodiment(s) of this specification as suitable. In other words, features between the various exemplary embodiments described herein are interchangeable as suitable, and not exclusive.

To address the foregoing challenges, the present disclosure provides three-dimensionally printed, wearable, electronic, sensing articles, particularly which may further be wireless, battery-free, non-invasive and personalized to the body of the wearer and entirely digitally manufactured. The sensing article may particularly comprise an elastomeric base (backbone/platform) structure, comprising a mesh, formed of one or more 3D printed elastomeric polymer compositions to provide a base/mesh which is flexible, soft and stretchable (elastic), whereby the base/mesh is conformable (e.g. circumferentially) to changes in body shape of the wearer's body during movement, particularly by elastic deformation. 3D printing enables digital control over geometry, allowing for tailored designs, topologies, and sensor locations that are specific to diagnosis and patient needs. The elastomeric base/mesh provides favorable mechanical properties similar to the epidermis, enabling intimate contact of sensor nodes to the body and increasing signal fidelity due to lowered skin impedance (thermal, electrical, and mechanical). Embedded/encapsulated in the elastomeric base/mesh is a soft antenna structure that incorporates a far-field energy harvesting system that operates at 915 MHz, a widely used frequency for wireless power transfer systems approved by the United States Federal Communications Commission.

While portions of the sensing articles described herein are described as being three-dimensionally printed, such should be understood as being preferred, particularly when the article is personalized to the body of the wearer, i.e. formed with photogrammetric mapping. However, it should be appreciated that the articles do not necessarily need to be personalized to the body of the wearer or three-dimensionally printed. For example, the portions of the sensing articles described herein as being three-dimensionally printed may be injection molded using thermoplastic elastomer, or laser cut from thermoplastic elastomer sheet (e.g. to form the mesh herein). Moreover, the articles may be produced in different sizes (e.g. small, medium and large) to fit bodies of different size.

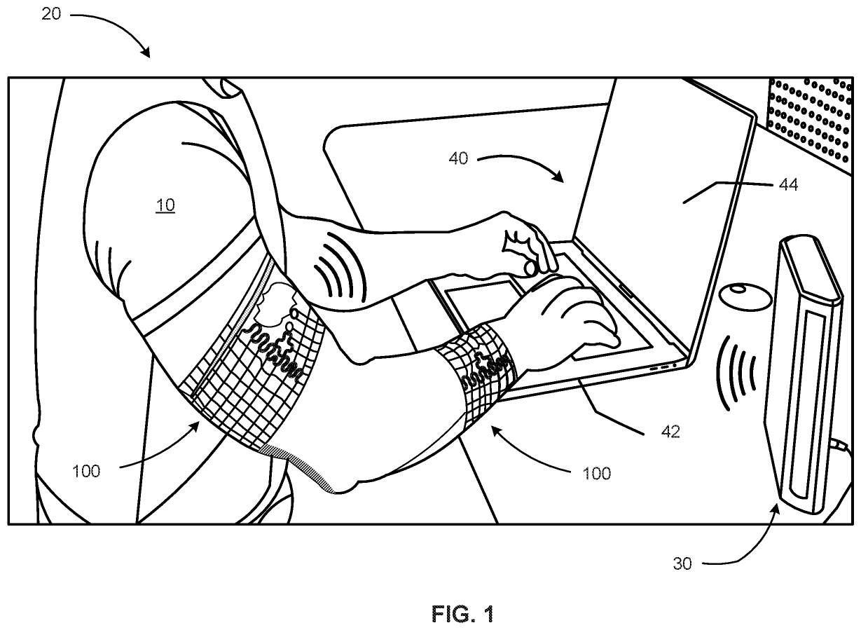

Referring to FIG. 1, there is shown a system 20, and particularly a healthcare monitoring and/or diagnostic system, according to the present disclosure. As shown, the system 20 comprises a wireless power caster/transmitter device 30, a sensing (biosymbiotic) article 100 (disposed on a human (host) body 10) and a remote wireless electronic (data collection) device 40. As shown, the remote electronic (data collection) device 40 may be a computer 42 such as a desktop computer, a portable computer such as a laptop computer, a notebook computer, a netbook (with or without a keyboard such as an iPad), a personal digital assistant (PDA), a cellphone computer or other handheld computer (e.g. smartphone). Computer 42 includes a visual display 44, which may be understood as a computer output surface and projecting mechanism that shows text and often graphic images (e.g. as part of a graphical user interface) to the computer user (which may be the host of the sensing article 100), such as via a liquid crystal display (LCD), light-emitting diode (LED), gas plasma (GS), or other image projection technology. The display 44 may be a touch activated display.

The sensing articles 100 disclosed herein are three-dimensionally (3D) printed, wearable, electronic, sensing articles 100, particularly to be worn directly on the epidermis (and hence may also be considered epidermal). The sensing articles 100 may also be wireless, battery-free, non-invasive and personalized to the body 10 of the wearer.

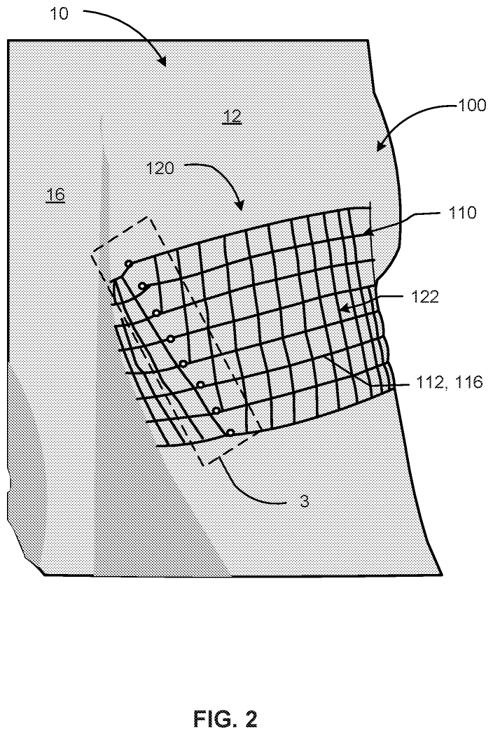

As discussed in greater detail, infra, a sensing article 100 is three-dimensionally (3D) printed in a two-dimensional (planar) form, and may be subsequently wrapped circumferentially around the body 10, particularly fully around (i.e. 360 degrees) an appendage 12 of the body 10 such as an arm or leg, or the torso 16. As shown in FIG. 2, the sensing device 100 is wrapped around the upper arm, particularly around the area of the bicep and tricep.

As shown in FIGS. 2-4, the sensing article 100 comprises a 3D printed elastomeric platform base 110, particularly in a form of a mesh 112 comprising a plurality of intersecting (e.g. transverse) strut segments 116 arranged in a lattice structure 120, particularly in which at least some of the strut segments 116 are arranged substantially parallel or perpendicular (e.g. in a range of 0.1 degrees to 10 degrees of being parallel or perpendicular, and more particularly in a range of 0.1 degrees to 5 degrees of being parallel or perpendicular) to one another as to create a grid of polygonal (quadrilateral and more particularly a rectangular or square) shaped window openings 122.

The individual strut segments 116 may have a cross-sectional shape which may be polygonal (e.g. quadrilateral and more particularly rectangular or square). The cross-sectional area of the individual strut segments 116 may be in a range of 0.06 mm² to 1 mm², and more particularly in a range of 0.16 mm² to 0.64 mm². The window openings 122 may have an area in a range of 16 mm² to 225 mm², and more particularly in a range of 36 mm² to 144 mm². The base 110/mesh 112/struts 116 may be made of thermoplastic elastomer, particularly thermoplastic polyurethane.

As shown in FIGS. 2-3, opposing end regions 130, 132 of the base 110/mesh 112 may be joined at a seam 134. As shown, the seam 134 is formed by cantilevered strut segments 116a of end region 130 which are connected in one-to-one relationship to cantilevered strut segments 116b of end region 132. The pairs of connected strut segments 116a, 116b may be joined by first placing the unconnected pairs of strut segments 116a, 116b side-by side such that at least a portion of their longitudinal lengths overlap. Thereafter, the overlapping portions may be heated, melted, pressed together and subsequently cooled (e.g. melt bonded) to join the pairs of strut segments 116a, 116b and end regions 130, 132 together at head/joint 136. In the foregoing manner, the end regions 130, 132 of the base 110/mesh 112 are mechanically integrally connected monolithically (i.e. from the material of the one-piece base 110/mesh 112 without need for separate fasteners). However, it should be understood that connection of the end regions 130, 132 is not limited to the foregoing, and the end regions 130, 132 may be connected with any suitable permanent (non-releasable) or releasable fasteners (e.g. heat shrink tubing, butt connectors, splice connectors, hook and loop connectors).

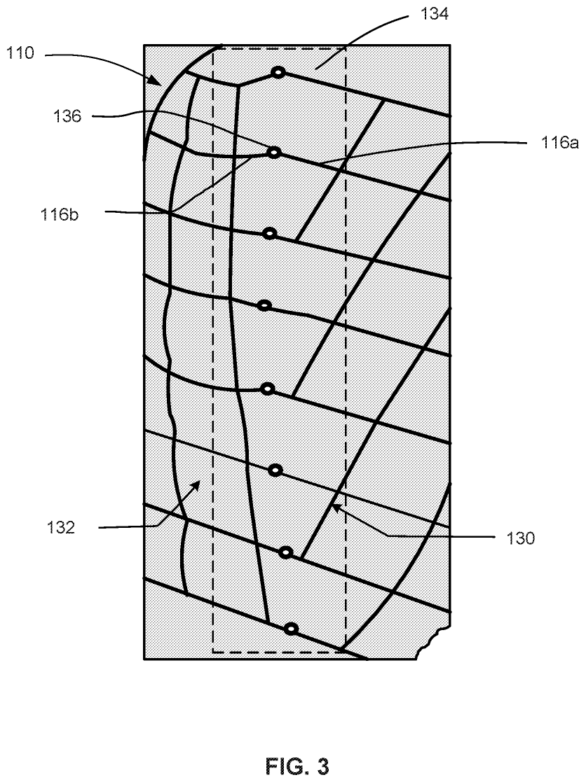

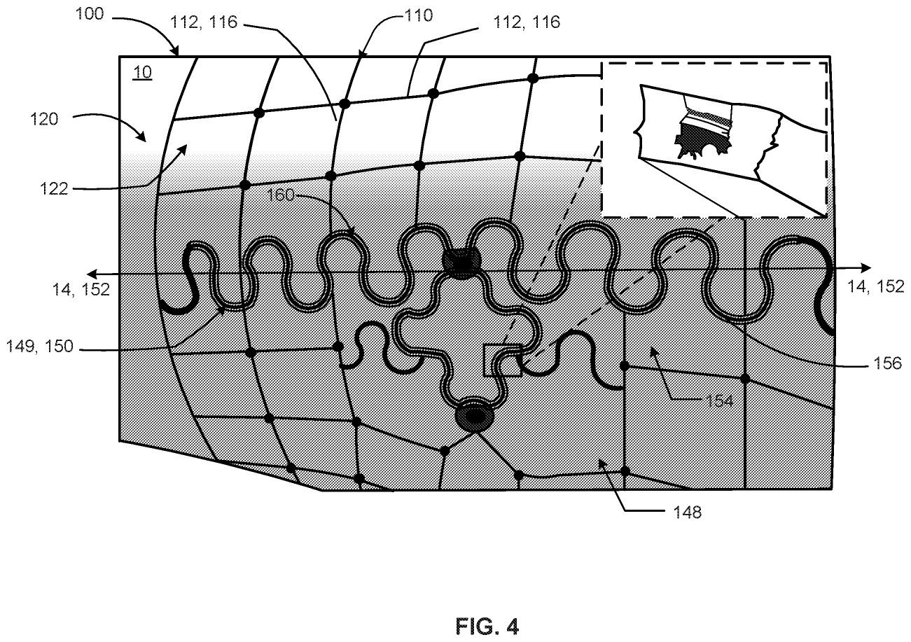

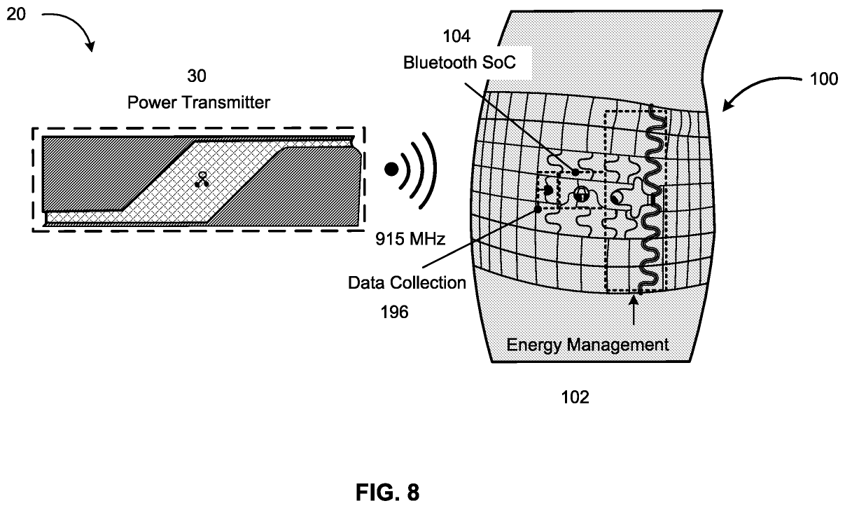

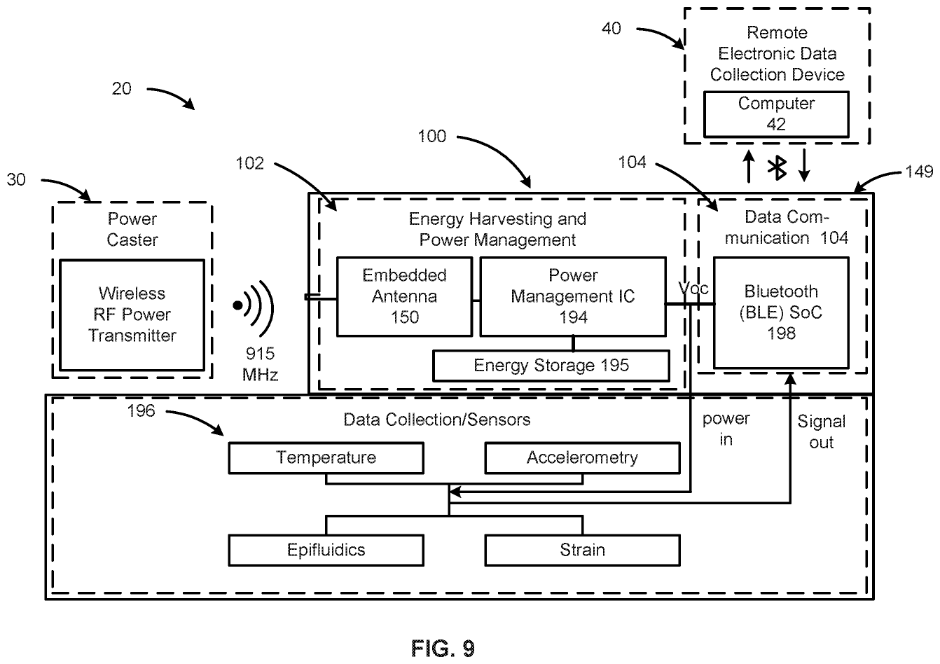

Now referring to FIGS. 4-6B, the article 100 may further comprise an electronic structure 148 which comprises an electronic component 149 in a form of an antenna 150, and more particularly a wireless energy harvesting antenna 150, which also may be referred to as a rectenna or rectifying antenna, which operates as part of a far-field (radio frequency), wireless, energy harvesting/power management device 102 (see FIGS. 8-9) of article 100. As explained in greater detail, infra, the antenna 150 harvests energy from the power caster device 30, particularly to power electronic components 149 of the sensing article 100 which consume power (e.g. data collection sensors 196).

As shown, the antenna 150 comprises an antenna structure having a curvilinear (serpentine/oscillating) repeating wave shape 154 which may extend in an antenna longitudinal direction 152, which, as shown, is also a transverse direction relative to the sensing article longitudinal direction 158. When sensing article 100 is disposed on the body 10 (e.g. extending/wrapped around an appendage 12), the antenna longitudinal direction 152 extends along the longitudinal (proximal-distal) direction 14 of the appendage 12, while the sensing article longitudinal direction 158 extends in a circumferential direction around the appendage 12. As shown, the wave shape 154 is formed by a plurality of alternating, reverse semi-circular (e.g. in a range of 180-200 degrees) segments 156 arranged adjacent one another end to end in the antenna longitudinal direction 152.

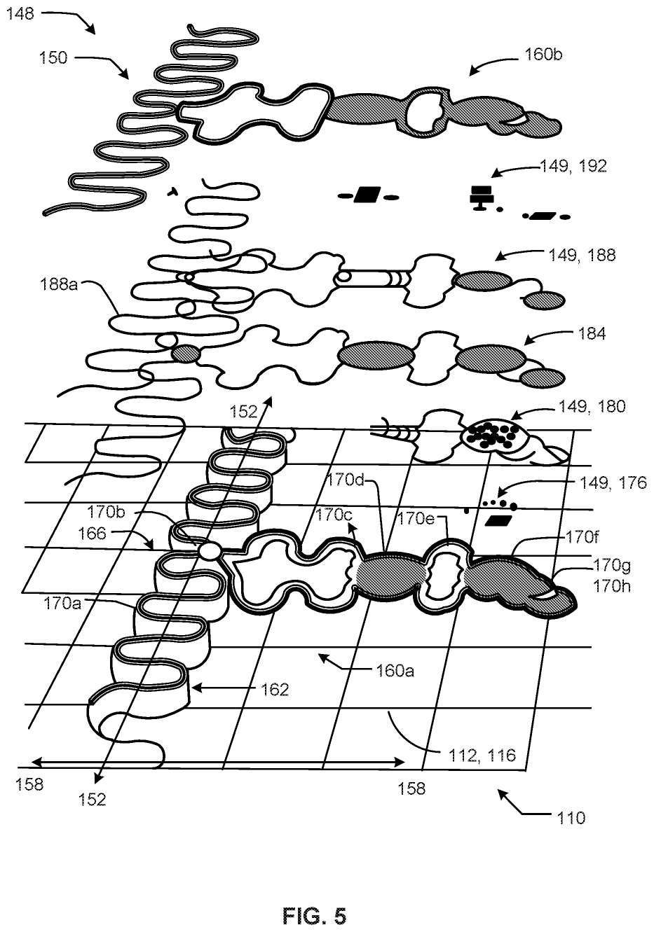

The antenna electronic components, along with other electronic components 149 discussed in further detail, infra, are at least partially embedded/encapsulated, and more particularly preferably fully embedded/encapsulated, within a three-dimensionally 3D printed electronic component enclosure 160 of the sensing article 100, which is shown in FIG. 5 as a lower enclosure 160a and an upper enclosure 160b for purposes of illustration.

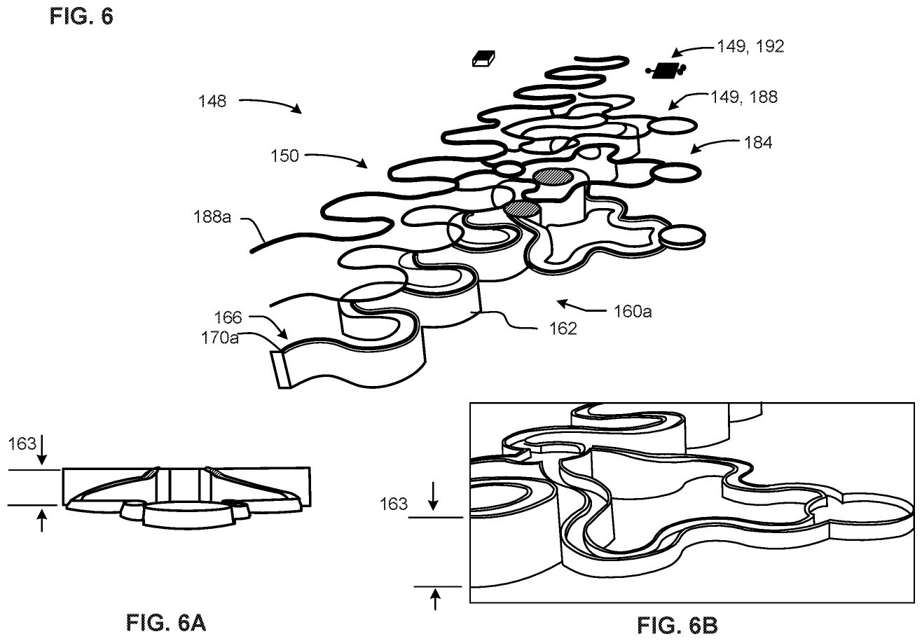

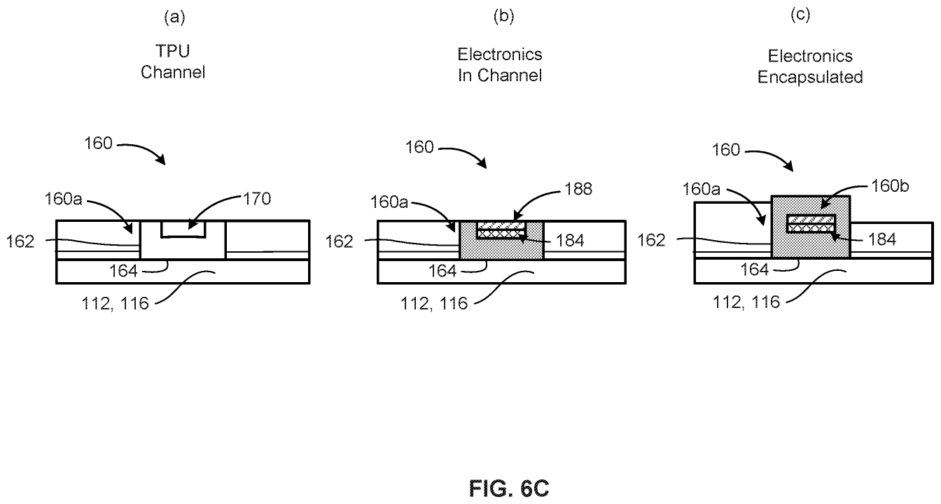

The electronic enclosure 160 may be formed of the same three-dimensionally (3D) printed elastomeric material as the base 110/mesh 112 and thus formed as one-piece with the base 110/mesh 112. The enclosure 160 may be in the same plane as the strut segments 116 of the base 110/mesh 112, in which case the strut segments 116 are connected laterally to a vertical side 162 of the enclosure 160, and to not extend underneath the enclosure 160. Alternatively, as shown in FIG. 6C, the enclosure 160 may be disposed on top of (i.e. raised above/overlie) the strut segments 116 of the base 110/mesh 112, in which case the strut segments 116 are connected to a bottom side 164 of the enclosure 160. Moreover, it should be understood that, in certain embodiment, certain strut segments 116 may be connected laterally to the vertical side 162 of the enclosure 160, while other strut segments 116 may be connected to the bottom side 164 of the enclosure 160.

During manufacture, the top side 166 of the lower enclosure 160a may be formed with a recess 170, which may be further divided into connected recesses 170a, 170b, 170c, 170d, 170e, 170f, 170g, 170h, to arrange and immobilize the various electronic components 149 of the sensing article 100 while the upper enclosure 160b is formed in situ on the lower enclosure 160a. As shown, recesses 170a, 170c, 170e and 170g are elongated grooves, while recesses 170b, 170d, 170f and 170h are circular nodes.

Once the lower enclosure 160a is formed with recess 170, a layered electronic structure may then be disposed in the recess 170. The layered structure, may comprise in ascending order lower electronic components 176, lower electrical power (flexible metal copper) conductor 180, electrical (flexible plastic polyimide) insulator 184, upper electrical power (flexible metal copper) conductor 188 and upper electronic components 192. Apart from the antenna electronic components, the electronic components 149, including components 176, 192, as discussed in further detail, infra, may include data collection sensors such as temperature sensors, accelerometers, epifluidic (sweat) sensors and strain gauge sensors; an AC to DC rectifier; a power management integrated circuit (IC); an energy storage device, such as a super capacitor and/or a battery; and a System on a Chip (SoC) with wireless (Bluetooth) communication. Thus, the electronic enclosure 160 may further comprise, for example, an antenna enclosure, a temperature sensor enclosure, an accelerometer enclosure and epifluidic sensor enclosure and a strain gauge enclosure, an AC to DC rectifier enclosure; a power management integrated circuit (IC) enclosure; an energy storage device enclosure, such as a super capacitor and/or a battery enclosure, and a System on a Chip enclosure. It should be understood that all the foregoing structures are not necessarily required. For example, as shown in FIG. 6, in certain embodiments the lower electronic components 176 and lower electrical power (flexible metal copper) conductor 180 may be eliminated.

Once the foregoing structures are disposed in recess 170, the upper enclosure 160b is formed in situ on the lower enclosure 160a, whereby the contents of the sensing article 100 disposed in the recess 170 are now fully embedded/encapsulated in a closed cavity (see inset of FIG. 4).

As best shown by FIGS. 6A-6B, certain electronic components 149 may be vertically raised in the enclosure 160 relative to other electronic components 149, such that the electronic components 149 are further away from the body 10 when the sensing article 100 is disposed thereon. More particularly, the antenna (power receptor) segment 188a of the electrical power conductor 188 is vertically raised away from the body 10, particularly by a vertical height 163 of the lower enclosure 160a. The antenna (power receptor) segment 188a is raised away from the body 10 to reduce electromagnetic interference from the body 10 from inhibiting power transmission to the antenna 150.

In FIG. 6C, the lower structure 160a disposed on top of (i.e. raised above/overlie) the strut segments 116 of the base 110/mesh 112, in which case the strut segments 116 are connected to a bottom side 164 of the lower enclosure 160a.

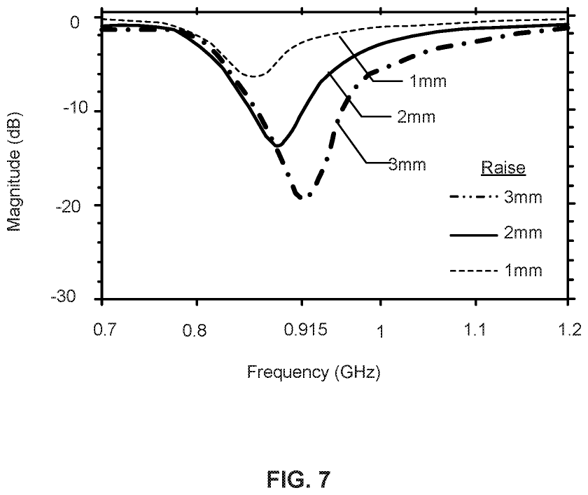

FIG. 7 shows a measurement of the performance of the antenna 150 of the sensing article 100 on the body 10, particularly a wrist. More particularly, FIG. 7 shows simulation results when the antenna (power receptor) segment 188a of the antenna 150 is raised to different heights 163 (i.e. raised levels) in a range of 1 mm to 3 mm from the body 10. 3D printing of topologically complex structures of the sensing article 100 enables the implementation and improved performance of the antenna 150.

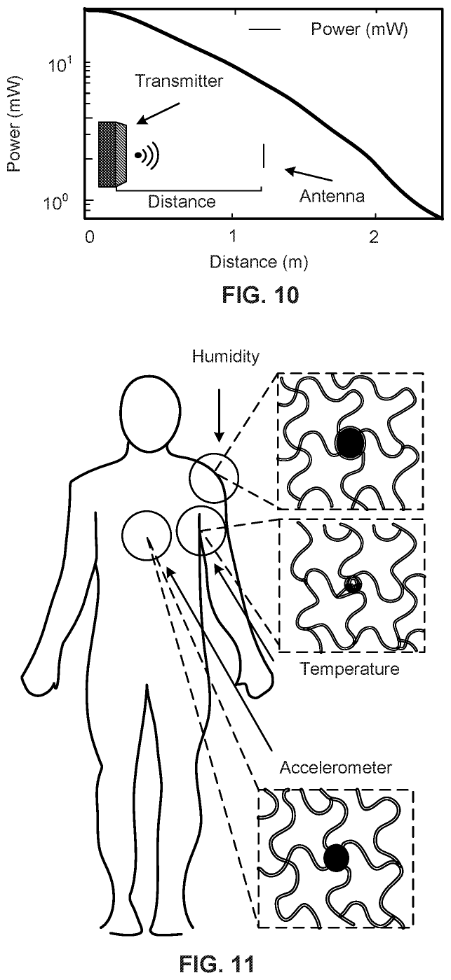

Referring now to FIGS. 8-10, applied together with the foregoing description and figures, the sensing article 100 provides a robust energy harvesting platform device 102, comprising an antenna 150 and other electronic components 149 comprising a power management integrated circuit 194 and an energy storage device 195 (e.g. supercapacitor, battery) to power further electronic components 149 which may comprise data collection sensors 196 and a short-range wireless communication transmitter and receiver device 104 (e.g., 10 meters or less) comprising a Bluetooth-enabled system-on-a-chip (SOC) 198 over a distance of a few meters (see FIG. 10) without the need for a battery.

The electronic components 149 are embedded in the 3D printed sensing article 100, and more particularly the 3D printed electronic enclosure 160, using discrete nodes (e.g. 170b, 170d, 170f and 170h) with a laser structured, ultra-flexible circuit substrate (e.g. 180, 184, 188) to maintain favorable mechanical properties. These electronic components 149 are embedded into the 3D printed sensing article 100, and more particularly the 3D printed electronic enclosure 160, in a one-piece monolithic platform, simplifying the production process using methods compatible with reel-to-reel manufacturing, allowing for rapid scalability. FIG. 8 shows the integration and relative locations of the electronic components 149 including the antenna 150 and other electronic components 149 embedded into the 3D printed sensing article 100, and more particularly the 3D printed electronic enclosure 160, which has a high tolerance for strain. A high-level operational schematic of the system 20 is shown in FIG. 9. The system 20 harvests RF power from a commercially available 915 MHz power caster 30. The energy is converted via a rectifier to direct current (DC) power and is stabilized by a power management device 102 that enables long-range functionality even when the power harvested is not constant. The energy is used to power the Bluetooth SOC 198 and other active electronic components 149 including the sensing modalities. Data is collected from sensing nodes placed strategically in the 3D printed sensing article 100, and more particularly the 3D printed electronic enclosure 160, and information is streamed to a Bluetooth-enabled device (e.g. computer 42) for storage and analysis. Sensing capabilities include sensors 196 such as a sub-millikelvin resolution temperature sensor, a relative humidity sensor with 2% accuracy, low noise inertial measurement unit, and 3D printed strain gauge. With low-power, highly miniaturized node footprints that are less than 6 mm in diameter, an imperceptible visual and mechanical feel may be achieved, while attaining high-fidelity signal recordings through a low impedance interface to the skin. To characterize and qualify the mechanical and chemical properties of the article/components for continual use on patients, a benchtop platform may be used to determine the mechanical characteristics of complex designs as well as long-term robustness before clinical trials through accelerated stress tests in physiological solutions. Data collected using this system 20 will guide design considerations to improve the longevity of the article/components and enhance the robust mechanical characteristics needed for long-term data acquisition.

The 3D printed, wearable, electronic, sensing article 100 is highly customizable with respect to sensing modalities and demonstrated sensing modalities include, accelerometry, temperature, and skin humidity. Preliminary testing has demonstrated a robust ability to acquire clinical-grade data streams over an extended period. Preliminary measurements of core body temperature, local skin humidity, and trunk motion have been collected using prototyped articles.

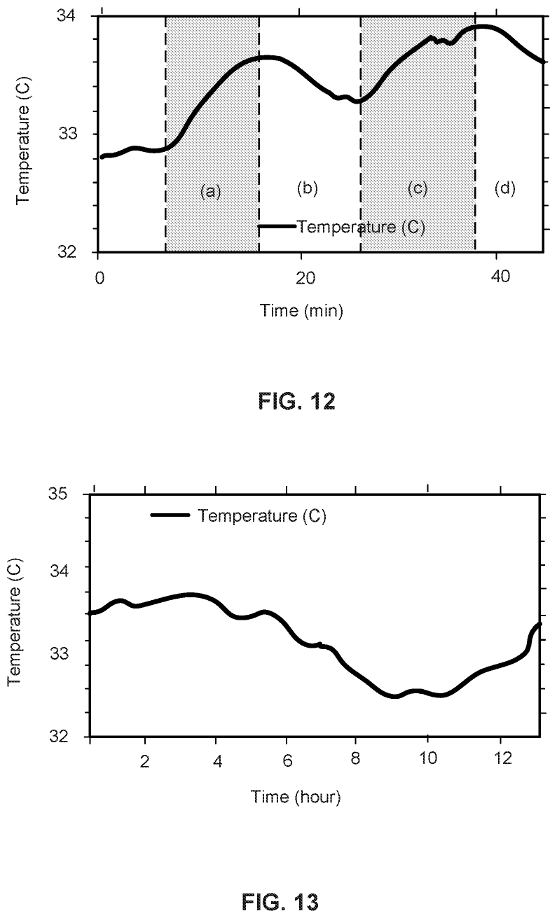

FIG. 11 shows some of these sensing modalities embedded in the article 100. The ultra-sensitive, low powered temperature sensor that features a 0.004 C resolution over the physiological range for surface body temperature in the under-arm. The mathematical model used for data correlation provides a less than 1% difference between theoretical and experimental system values, yielding high confidence in the data captured by this system. FIG. 12 shows the responsiveness and sensitivity of the sensor. In this experiment, the subject wore the article for 50 minutes during which they performed 8 minutes of light activity (zones (a) and (c)) followed by 10 minutes of rest (zones (b) and (d)). From the data, periods of heighted activity may be discerned and changes in steady-state body temperature after each sequential period of activity. In FIG. 13, the subject wore the article 100 for 13 hours, from 6 pm to 7 am. In this experiment, clear periods of heighted activity denoted by short-term increases in recorded temperature values were observed. The long-term trend of the data indicates the ability of the sensors to detect subtle changes in homeostasis such as the circadian rhythm.

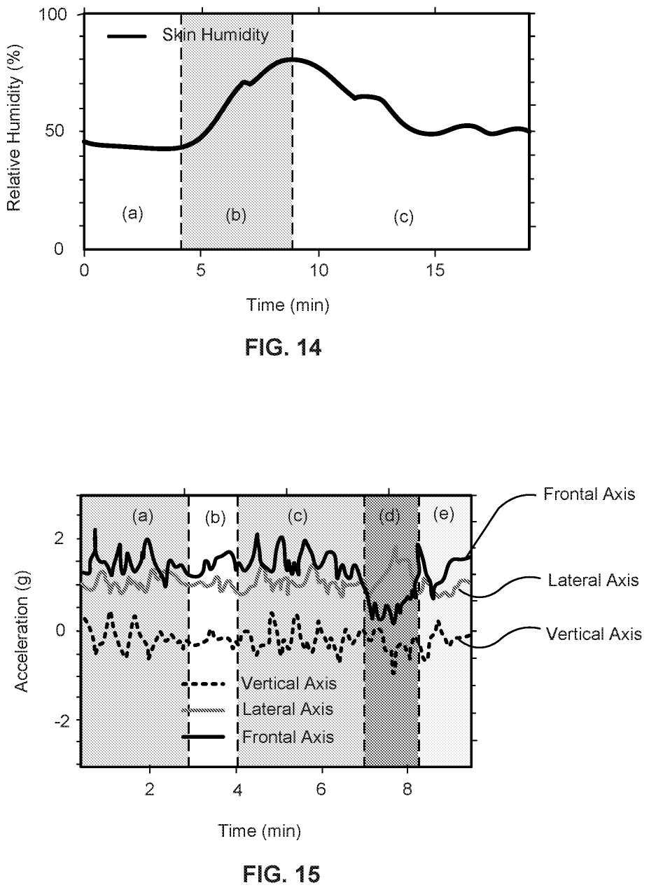

Relative skin humidity is another physiological parameter that may be monitored to identify the onset of health concerns and acute physiological events. The data can also be used to monitor patient activity in conjunction with temperature and acceleration, while allowing for unique insight into previously unmeasured physiologies that could supplement the evaluation of patient health and activity. Increases in skin humidity and perspiration may aid in the detection of acute illness and provide semi-quantitative values of a patient's physical strain during activity. To monitor these characteristics, a low-power, highly miniaturized relative humidity sensor situated on the distal region of the upper shoulder may be used. The sensor node, which is 5 mm in diameter, utilizes 3D-printed topologies to allow for a localized influx of air to the sensor without compromising the electronics embedded in the article 100. Initial testing revealed high responsiveness of the sensor platform located on the distal portion of the shoulder. During the experiment depicted in FIG. 14, the subject wore the system for 20 minutes, in which they began by establishing a baseline during a 3-minute resting phase (zone (a)), performed activities including moderate walking and climbing stairs (zone (b)) for 6 minutes, and then rested again (zone (c)). The system demonstrated enhanced capabilities for detecting periods of heightened exercise.

Additionally, accelerometry has been demonstrated, as it will provide the meta data needed to analyze gait. A low-power accelerometer unit has been tested to measure trunk motion using the article 100. Our accelerometer platform is 5 mm in diameter and is embedded in a fixed orientation, enabling robust acceleration measurements in axis orientations that are temporally consistent. Preliminary data for this system is displayed in FIG. 15. In this experiment, the subject wore the sensing platform attached to their trunk (i.e. torso), and then performed 3 seconds of walking (zone (a)) followed by a 180 degree turn (zone (b) and 3 additional seconds of walking (zone (c)). The subject then knelt forward (zone (d)) and then returned to their original standing position (zone I). From the data, periods of activity could be distinguished and it could be determined when the subject knelt and stood up from changes in the lateral and vertical axis acceleration. The sensitivity of this modality, paired with the intimate epidermal contact, may provide clinicians and statisticians with the high-quality data needed to perform frailty analysis for this study.

Thus, a wireless, battery-free, 3D printed sensing article 100 suitable for recording high-fidelity, clinical-grade, and continuous data streams to allow for advanced diagnosis using an imperceptible form factor is provided. While the data shown here was recorded on single mode devices, it is feasible to combine the modalities into one platform to obtain a holistic view of patient activity and physiology over time.

To provide the three-dimensionally printed, wearable, electronic, sensing article 100, which is also wireless, battery-free, non-invasive and personalized to the body of the wearer, highly scalable digital manufacturing techniques may be utilized, which further allow for rapid reconfiguration of the sensing system based on target biomarker which may be personalized to the individual subject in the study. Critical to a personalized sensing article 100 that is applied epidermally is an intimate fit of localized sensors directly at the site of interest. This ensures minimal motion artifacts which are induced by relative motion of sensor and epidermis. Moreover, the article 100 may be applied without use of an adhesive, and hence not suffer from associated poor chronic sensor performance after renewal of the epidermis, which occurs depending on subject, after 1-2 weeks and irritation that occurs even with the use of clinically tested adhesives.

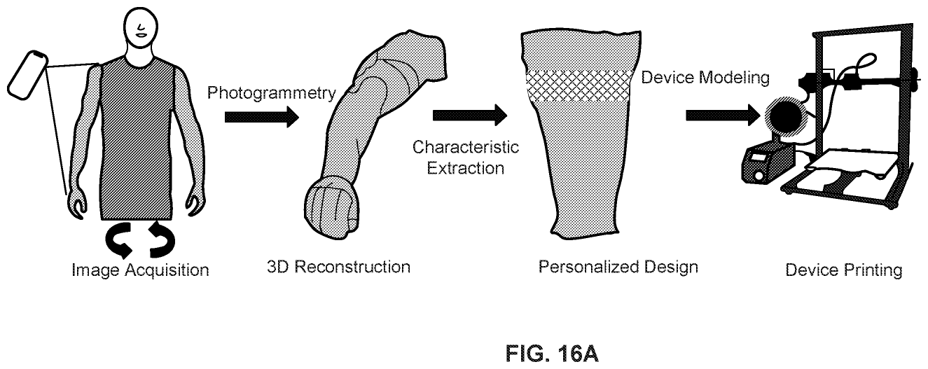

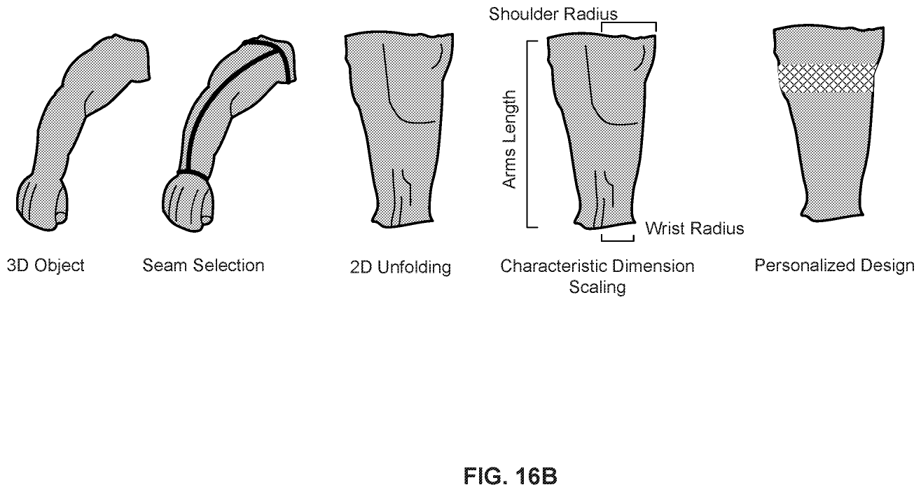

Referring to FIGS. 16A-16B, such depict formation of an article 100, and more particularly a base 110 particularly in the form of the mesh 112, which is personalized to the body 10 of the wearer. As shown, to generate the personalized article 100, at least a surface of a selected region of a body 10 of a particular (unique) wearer of the article 100 is three-dimensionally, digitally mapped. For example, as shown, at least the surface of an appendage 12 (e.g. arm) of the body 10 of the particular wearer may be obtained by photogrammetric mapping, which may simply be referred to as photogrammetry. Other 3D digital mapping techniques, such as magnetic resonance imaging (MRI) and computerized tomography (CT) scanning, may also be utilized as appropriate. Next, a 3D digital model (mathematical representation) of the surface of the selected region of the wearer's body 10 is generated from data of a dataset of the 3D mapping. Data from the dataset of the 3D digital model of the surface of the selected region of the wearer's body 10 is then manipulated, via software (particularly by unfolding the data of the 3D model dataset), to generate a two-dimensional (2D) template for three-dimensional printing.

Three-dimensional printing (e.g. fused filament fabrication (FFF); fused deposition modeling (FDM)) of the base 110 of the article 100 is next performed, from the two-dimensional template, where the base 110 of the article 100 comprises the mesh 112, which is formed of an elastomeric polymer composition. By virtue of being formed of the elastomeric polymer composition, the base 110/mesh 112 is flexible, soft and stretchable (elastic), whereby the base 110/mesh 112 is conformable (e.g. circumferentially) to changes in body shape of the wearer's body 10, and more particularly the surface of the selected region of the body 10 of the wearer of the article 100, during movement. Moreover, the individually tailored fit results in at least substantially perfect conformality to the wearer's body 10, enabling high-fidelity and chronic biodata acquisition that is adhesive free, which may enable continuous attachment to the wearer's body for periods of duration at least a week (e.g. at least 2 weeks).

The elastomeric polymer composition may be at least one of a synthetic elastomeric polymer composition and a thermoplastic elastomeric polymer composition, which may comprise, essentially consist of, or consist of, at least one elastomer, which may comprise, essentially consist of, or consist of at least one of a synthetic polymer and a thermoplastic polymer.

The elastomeric polymer composition may have a glass transition temperature (Tg) below 23° C. and be, at most, 50% crystalline (i.e., the composition contains an amorphous phase of 50% or greater, up to 100% amorphous phase). Additionally, or alternatively, the elastomeric polymer composition may be a polymer composition that has an elongation at 23° C. of at least 100%, and which, after being stretched to twice its original length and being held at such for one minute, may recover in a range of 50% to 100% within one minute after release from the stress. More particularly, the elastomeric polymer composition may recover in a range of 75% to 100% within one minute after release from the stress, and even more particularly recover in a range of 90% to 100% within one minute after release from the stress. Additionally, or alternatively, the elastomeric polymer composition may have the following mechanical properties.

| Mechanical Property | Test Method | Value (range) |
|---|---|---|
| Shore A Hardness | ASTM D2240 | 60-100 Shore A |
| Tensile Strength | ASTM D412 | 2-60 Mpa |
| Elongation @ Break | ASTM D412 | 400-800% |

Note:
The foregoing ASTM test methods is the most recent test methodadopted by the ASTM as of the data of filing the application.

In one particular embodiment the elastomeric polymer composition may be a thermoplastic urethane (TPU) elastomeric polymer composition, wherein the at least one elastomer may be a thermoplastic urethane (TPU) elastomer. The thermoplastic urethane (TPU) elastomer may be supplied by, for example, NinjaFlex by NinjaTek, Varioshore by Colorfabb, X60A by Diabase Engineering.

Thus, the present disclosure provides an article 100 that can be created using a set of smart phone pictures and photogrammetry to yield body shape to automatically create a printable, soft and wearable mesh design that can house a cohort of sensors, radio frequency (RF) energy harvesting antenna for indefinite operation at distance, and Bluetooth radio to relay sensor information.

The article 100 may provide intimate epidermal contact that enables high-fidelity extraction of biophysical parameters such as, but not limited to, temperature with mK resolution, 9 axis inertial measurement unit data and skin humidity to detect sweat onset before liquid sweat secretion. Coupled with these biophysical parameters will be an inline electrochemical sweat detection platform that enables facile extension to broad classes of biomarkers using rapid, label-free, highly sensitive all-printed sensor arrays.

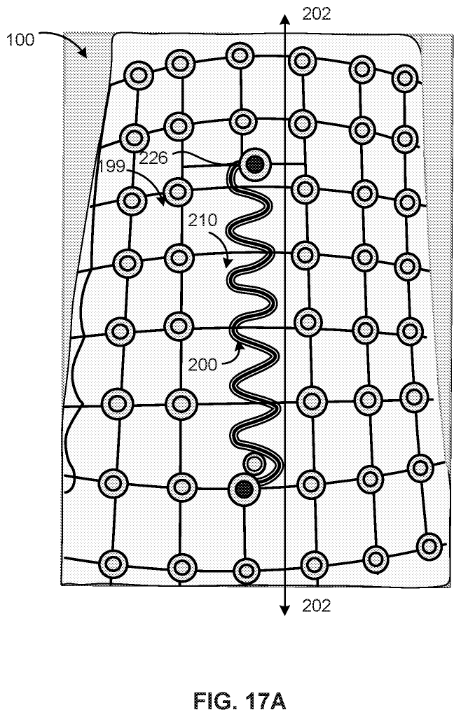

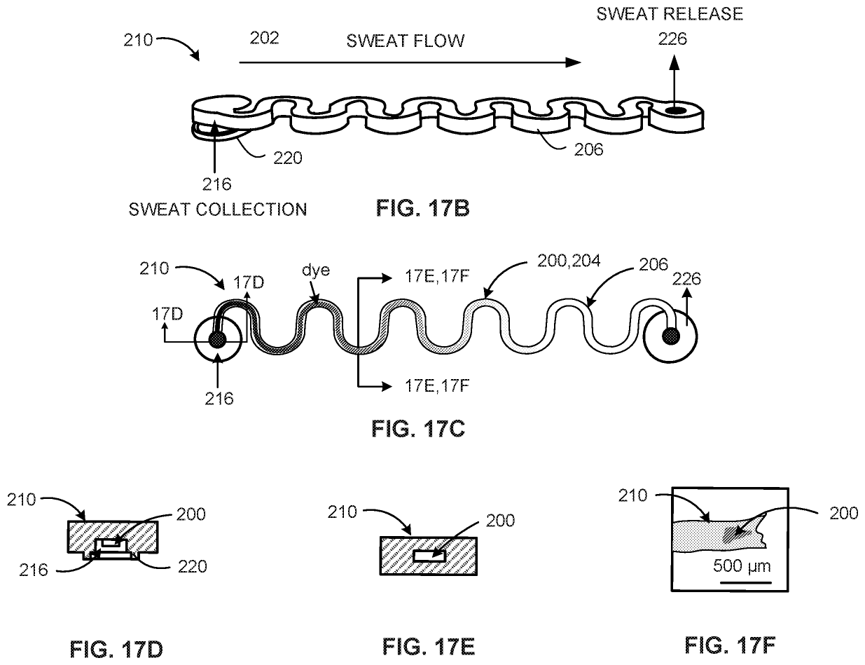

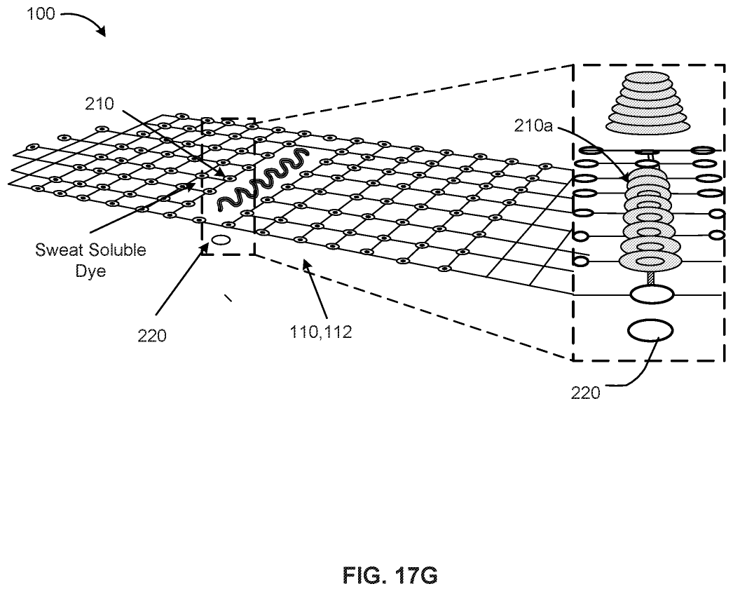

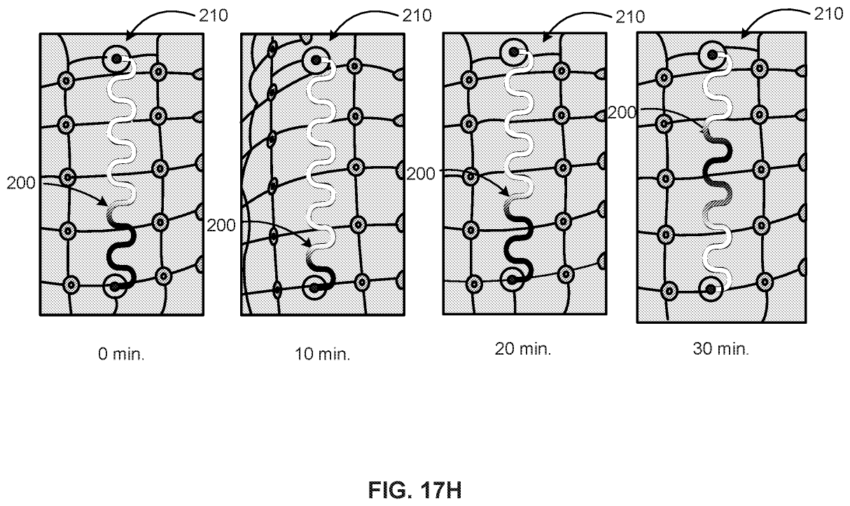

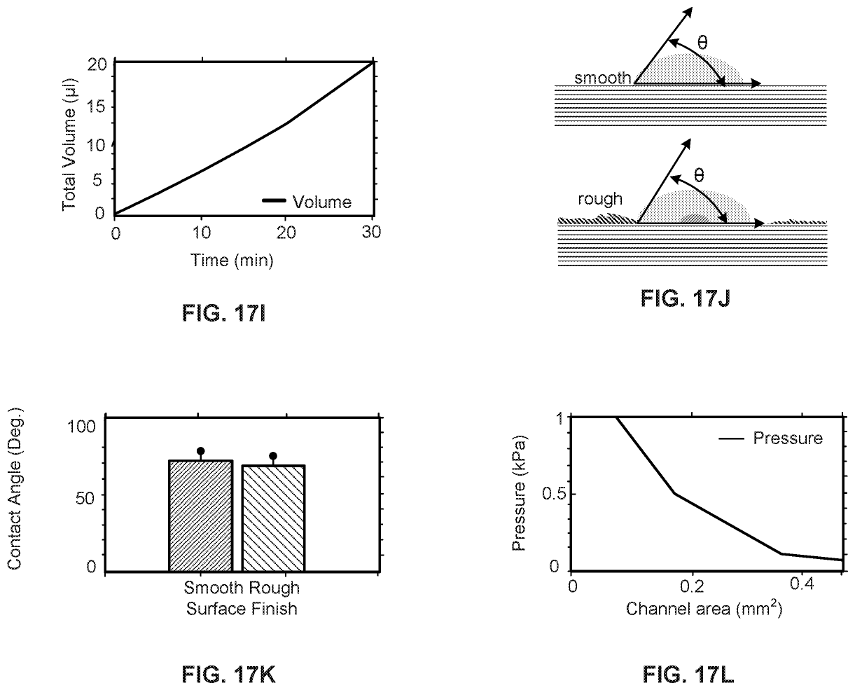

As shown in FIGS. 17A-17G, sensing article 100 may include a microfluidic structure 199, particularly including a microfluidic channel 200 in a microfluidic channel enclosure 210. Similar to the antenna 150, the microfluidic channel 200 may have a curvilinear (serpentine/oscillating) repeating wave shape 204 which may also extend in a microfluidic channel longitudinal direction 202, which is also a transverse direction relative to the sensing article longitudinal direction 158. When sensing article 100 is disposed on the body (e.g. extending/wrapped around an appendage 12), the microfluidic channel longitudinal direction 202 may extend along the longitudinal (proximal-distal) direction 14 of the appendage 14, while the sensing article longitudinal direction 158 extends in a circumferential direction around the appendage 12. As shown, the wave shape 204 is formed by a plurality of alternating, reverse semi-circular (e.g. in a range of 180-200 degrees) segments 206 arranged adjacent one another end to end in the microfluidic channel longitudinal direction 202. The wave shape 204 may provide a channel 200 having a desired overall length which is less than the corresponding longitudinal length, thus reducing longitudinal packaging.

The microfluidic channel 200 may have a cross-sectional area which is defined by three-dimensionally (3D) printed microfluidic channel enclosure 210 of the sensing article 100 (shown divided in FIG. 17G as a lower enclosure 210a and an upper enclosure 210b and an annual rib/O-ring 220 for purposes of illustration).

The microfluidic channel enclosure 210 may be formed of the same 3D printed elastomeric material as the base 110/mesh 112 and thus formed as one-piece with the base 110/mesh 112. As with the electronic enclosure 160, the microfluidic channel enclosure 210 may be in the same plane as the strut segments 116 of the base 110/mesh 112, in which case the strut segments 116 are connected laterally to a vertical side of the enclosure 210, and to not extend underneath the enclosure 210. Alternatively, as with the electronic enclosure 160, the microfluidic channel enclosure 210 may be disposed on top of (i.e. raised above/overlie) the strut segments 116 of the base 110/mesh 112, in which case the strut segments 116 are connected to a bottom of the enclosure 210. Moreover, it should be understood that, in certain embodiments, certain strut segments 116 may be connected laterally to the vertical side of the enclosure 210, while other strut segments 116 may be connected to the bottom side of the enclosure 210.

The microfluidic channel 200 may have a circular cross-sectional shape (i.e. transverse to the flow direction), in which case the channel 200 may be understood as being cylindrical, or other cross-sectional shape, such as polygonal (e.g. rectangular cross-sectional shape). Similarly, the microfluidic channel enclosure 210 may have a circular cross-sectional shape (e.g. annular), in which case the enclosure 210 may be understood as being cylindrical, or other cross-sectional shape, such as polygonal (e.g. rectangular frame cross-sectional shape). Thus, the microfluidic channel 200 or the microfluidic channel enclosure 210 may be defined by a circular (cylindrical and/or annular) or polygonal (rectangular frame) wall/surface of the article 100.

The microfluidic channel 200 has a (sweat collection) inlet 216 and a (sweat collection) outlet 226 disposed at opposite ends of the channel 200. The inlet 216 faces the skin, while the outlet 226 faces opposite the inlet 216 away from the skin. The channel 200 has a length from the inlet 216 to the outlet 226 in range of 10-100 mm, and a channel (cross-sectional) area in a range of 0.2-0.4 mm², and preferably of a uniform cross-sectional area along its length. The wave shape 204 of the microfluidic channel enclosure 210 may increase the stiffness/rigidity of the microfluidic channel enclosure 210, as compared to a rectilinear channel enclosure, particularly to inhibit deformation and change of the cross-sectional area of the channel 200 under pressure.

To better ensure intimate skin contact and form a better seal, a 3D printed elastomeric annular rib/O-ring 220 (see e.g. FIGS. 17D and 17G) may be disposed between the skin (directly on the skin) of the subject and the inlet 216 to the channel 200. The elastomeric annular rib/O-ring 220 may have a (rib) height in a range of 0.1 mm to 2.5 mm, more particularly in a range of 0.3 mm to 1.5 mm and even more particularly in a range of 0.5 mm to 1.2 mm The elastomeric annular rib/O-ring 220 may have a (rib) width in a range of 0.1 mm to 2.5 mm, more particularly in a range of 0.3 mm to 1.5 mm and even more particularly in a range of 0.5 mm to 1.2 mm The elastomeric annular rib/O-ring 220 may have a diameter in a range of 2 mm to 8 mm and more particularly 3 mm to 6 mm.

While the microfluidic channel enclosure 210 may be formed, particularly after the lower enclosure 210a is formed, a colorimetric dye may be applied to the 3D printed surfaces forming the microfluidic channel 200 in be allowed to dry prior to formation of the upper enclosure 210b in situ on the lower enclosure 210a.

During operation, eccrine sweat (i.e. sweat from the eccrine glands) may be collected into the microfluidic channel 200. As shown particularly by FIGS. 17C and 17H, sweat from the subject collected into the microfluidic channel 200 at the inlet 216 may then travel in the microfluidic channel 200 and then ultimately be released from the outlet 226. The dye is used to visually show the progression of the sweat in the channel 200, which can then be recorded as a function of time to determine the level to which the subject is sweating/perspiring. As shown by FIG. 17I, the volume of sweat over 30 minutes may be approximately 20 μl.

Fluid (sweat) flow into the microfluidic channel 200 may be better facilitated depending upon the contact angle, which may be represented by θ, of the fluid on the surface of the channel 200 formed of the 3D printed elastomer, such as thermoplastic urethane (TPU).

Contact angle θ is a quantitative measure of the wetting of a solid by a liquid. Contact angle θ may be defined geometrically as the angle formed by a liquid at the three phase boundary where a liquid, gas and solid intersect. In terms of the thermodynamics of the materials involved, contact angle θ involves the interfacial free energies between the three phases given by the equation γLV cos θ=γSV−γSL, where γLV, γSV and γSL refer to the interfacial energies of the liquid/vapor, solid/vapor and solid/liquid interfaces, respectively. If the contact angle θ is less than 90 degrees the liquid may be said to wet the solid. If the contact angle is greater than 90 degrees the liquid is non-wetting. A zero contact angle θ represents complete wetting. When the surface of the microfluidic channel 200 is formed with the TPU, the contact angle θ of the fluid (sweat) thereon is advantageously less than 90 degrees. The lower contact angle θ, particularly less than 90 degrees, may be understood to lower the resistance to flow of the fluid (sweat) into the channel 200. As shown by FIGS. 17J and 17K, with the TPU, the contact angle θ is in a range of 70-80 degrees, somewhat regardless of the surface finish being smooth or rough.

Given that the TPU has a contact angle θ with the fluid (sweat) which is less than 90 degrees for the surface of the microfluidic channel 200, the TPU surface may be understood to be a hydrophilic surface.

Fluid (sweat) flow into the microfluidic channel 200 may also be better facilitated depending upon the cross-sectional area of the microfluidic channel 200. FIG. 17L shows how the fluid pressure required for the sweat to enter the microfluidic channel 200 decreases as a function of increasing cross-sectional area of the microfluidic channel 200. As shown, the when the cross-sectional area of the microfluidic channel 200 increase from 0.2 to 0.4 square millimeters (mm 2), the fluid pressure required for the sweat to enter the microfluidic channel 200 decreases from 0.5 to 0.05 kPa.

Depending on the cross-sectional area of the microfluidic channel 200, when the surface of the microfluidic channel 200 is hydrophilic, the microfluidic channel 200 may exhibit capillary action with regards to uptake of the fluid (sweat). The lower the contact angle θ (the more hydrophilic the surface), the higher the capillary pressure and thus the stronger the capillary action. The capillary action is desirable as the pressure to take the fluid (sweat) into the microfluidic channel 200 will less than the pressure applied to the fluid (sweat) to expel it from the body 10.

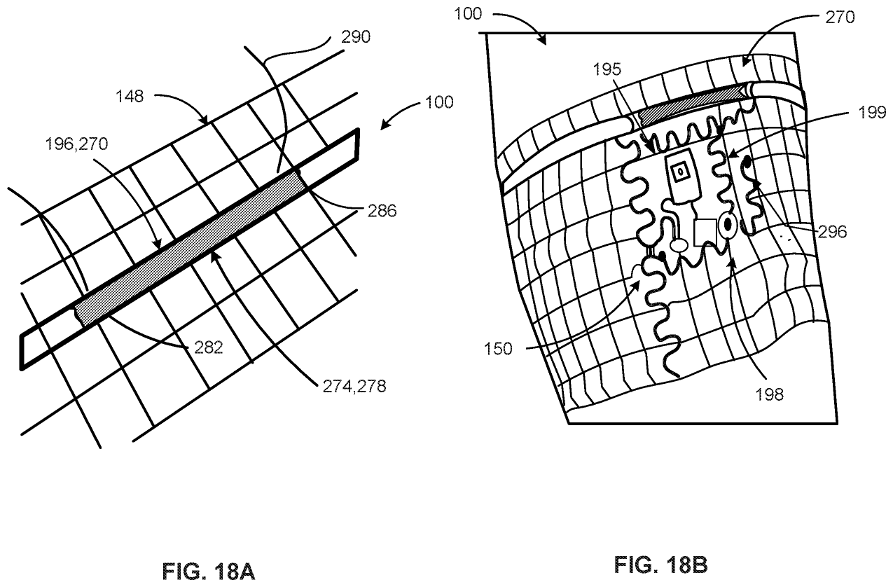

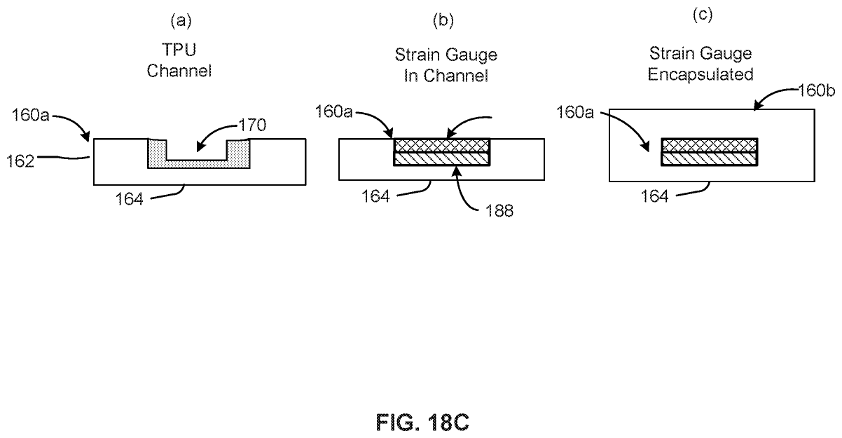

As shown in FIGS. 18A-18C, article 100 may include another electronic structure 148, particularly another sensor 196 in the form of a wireless stain gauge sensor 270. The strain gauge sensor 270 may be formed by placing electrical conductor 188 within recess 170 of the lower enclosure 160a. A length portion of the electrical conductor 188 may then be removed, in which case two (end) portions 282, 286 of the conductor 188 may now be electrically disconnected from one another. Thereafter, an elongated strain element 274 formed of electrically conductive thermoplastic elastomer (e.g. thermoplastic urethane filled with an electrically conductive particulate such as carbon black) may by three-dimensionally printed in/along the recess 170 at the location of the removed conductor 188 as well as overlie the two disconnected (end) portions 282, 286 of the conductor 188, thus reestablishing electrically connectivity of the conductor 188 along the length of the elongated strain element 274. The (end) portions 282, 286 of the conductor 188 may also be coupled to leads 290 to determine electrically resistance changes at opposing ends of the elongated strain element 274 in response to strain being applied to the elongated strain element 274 (e.g. due to contraction of muscles). As shown, article 100 also may include another sensor 196 in the form of a temperature sensor 296, as well as a microfluidics structure 199.

As shown by FIG. 18C, similar to the formation of antenna 150, once the lower enclosure 160a is formed with recess 170, a layered electronic structure may then be disposed in the recess 170. The layered structure, may comprise electrical power (flexible metal copper) conductor 188 and strain gauge sensor 270 disposed over the comprise electrical power (flexible metal copper) conductor 188.

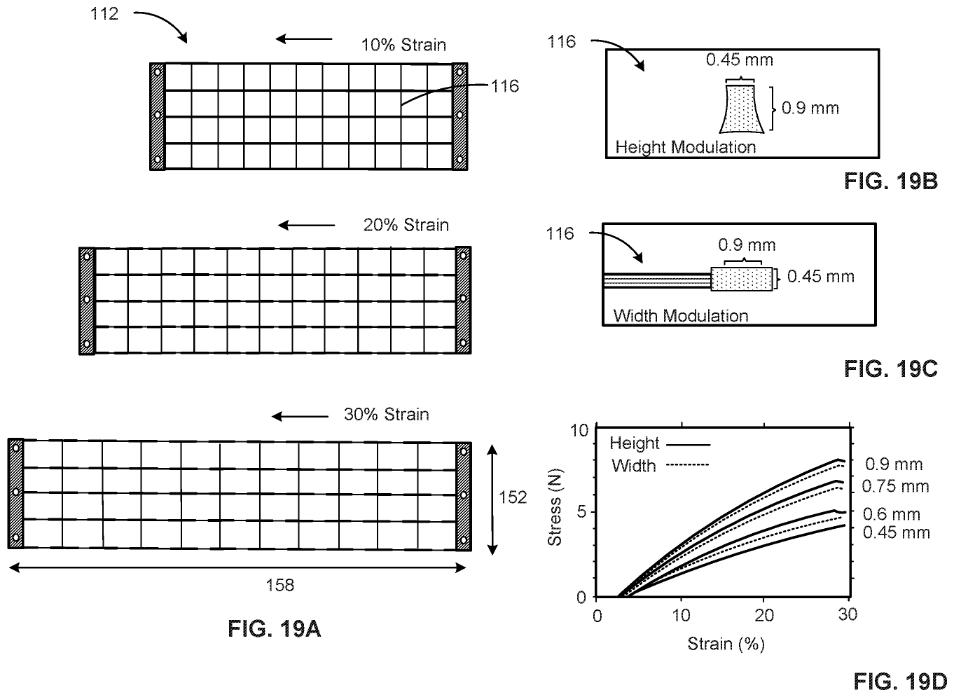

Referring now to FIGS. 19A-19D, there is shown some of the tunable bulk mechanics of the mesh. As shown in FIG. 19A, as strain increases from 10% to 30%, the struts 116 the longitudinal direction 158, the strain does not correspondingly increase in the struts 116 in the transverse direction 152. As explained, supra, such may be used to inhibit strain particularly on certain electronic components 149. FIGS. 19B and 19C shows tunability of the struts 116 by modulation of height and width. FIG. 19D shows stress versus strain curves displaying results of height and thickness modulation for the struts 116.

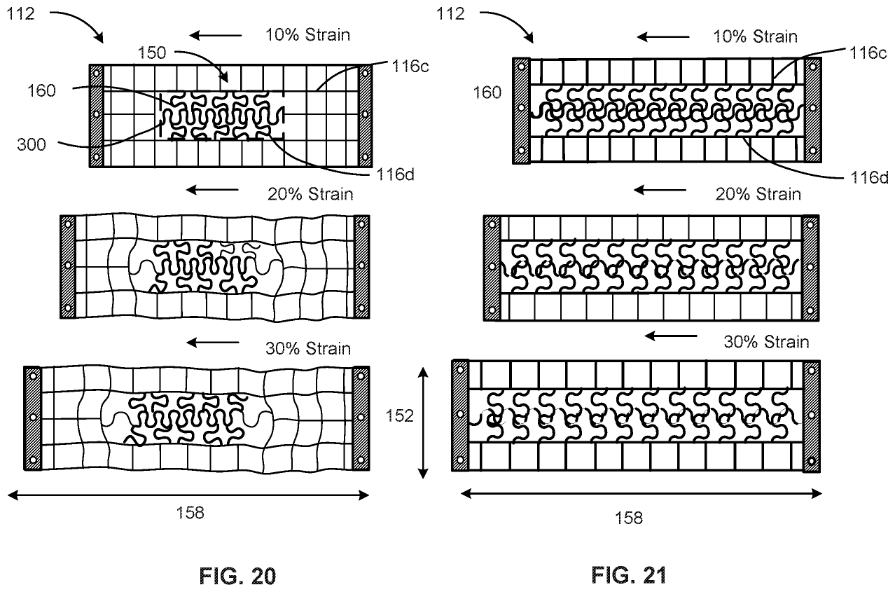

As set forth, supra, it may be desirable to arrange certain electronic components 149, such as the antenna 150, with the antenna longitudinal direction being the same as the sensing article transverse direction 152 as to reduce stress on the antenna 150 which may occur in the sensing article longitudinal direction 158, particularly when the sensing article 100 is wrapped around an appendage 12. However, as shown by FIG. 20, design of the struts 116 may be used strain isolate certain electronic components 149. As shown in FIG. 20, the antenna 150 may be arranged with the antenna longitudinal direction 152 being the same as the sensing article longitudinal direction 158.

As shown in FIG. 20, the strut segments 116 are connected laterally to a vertical side 162 of the enclosure 160, and to not extend underneath the electronic enclosure 160. As shown, all the strut segments 116 directly connected to the electronic enclosure 160, i.e. within region 300, are curved strut segments 116d (curvilinear along their respective lengths), particularly having a semi-circular curvature arc segment (e.g. in a range of 180-200 degrees). As shown, outside the surrounding region 300, the curvilinear strut segments 116d are directly connected to rectilinear strut segments 116c. In other words, one end of the curved strut segments 116d is connected to the electronic enclosure 160 while the opposing end is connected to a rectilinear strut segments 116c. Also as shown, the length of the curvilinear strut segments 116d between its opposing connection end points is greater than the length of the rectilinear strut segments 116c, respectively. The increased length of the curvilinear strut segments 116d as compared to the length of the rectilinear strut segments 116c provides for electrical component strain isolation.

As shown in FIG. 21, rather than the strut segments 116 being connected laterally to a vertical side 162 of the enclosure 160, the enclosure 160 (shown by a rectangular dashed line) may be disposed on top of (i.e. raised above/overlie) the curvilinear strain isolation strut segments 116d of the base 110/mesh 112, in which case the curvilinear strut segments 116d are connected to a bottom side 164 of the enclosure 160.

While particular embodiments of the present invention(s) has/have been described, it should be understood that various changes, adaptations and modifications can be made therein without departing from the spirit of the invention(s) and the scope of the appended claims. Further disclosure regarding the present invention(s) may be found in the publication of Stuart T, Kasper K A, Iwerunmor I C, McGuire D T, Peralta R, Hanna J, Johnson M, Farley M, LaMantia T, Udorvich P, Gutruf P. “Biosymbiotic, personalized, and digitally manufactured wireless devices for indefinite collection of high-fidelity biosignals” Science Advances 2021 Oct. 8; vol. 7(41):eabj3269. doi: 10.1126/sciadv.abj3269. Epub 2021 Oct. 8. PMID: 34623919; PMCID: PMC8500520, hereby incorporated by reference in its entirety. The scope of the invention(s) should, therefore, be determined not with reference to the above description, but instead should be determined with reference to the appended claims along with their full scope of equivalents. Furthermore, it should be understood that the appended claims do not necessarily comprise the broadest scope of the invention(s) which the applicant is entitled to claim, or the only manner(s) in which the invention(s) may be claimed, or that all recited features are necessary.

Listing of reference characters
10 body
12 appendage
14 appendage longitudinal direction
16 torso
20 system
30 power caster/transmitted device
40 remote electronic device
42 computer
44 display
100 article
102 energy management (power harvesting) device
104 transmitter and receiver device
110 base
112 mesh
116 strut segment
116a cantilevered strut segment
116b cantilevered strut segment
116c straight (rectilinear) strut segments
116d curved (curvilinear) strut segments
120 lattice structure
122 openings
130 end region
132 end region
134 seam
136 head
148 electronic structure
149 electronic component
150 antenna
152 antenna longitudinal direction (sensing article transverse direction)
154 antenna wave shape
156 antenna semi-circular segments
158 sensing article longitudinal (circumferential) direction
160 electronic enclosure
160a lower electronic enclosure
160b upper electronic enclosure
162 vertical side of enclosure
163 height of the lower enclosure
164 bottom side
166 top side
170 recess (170a-170h)
176 lower electronic components
180 lower electrical power conductor
184 electrical insulator
188 upper electrical power conductor
188a antenna (power receptor) segment
192 upper electronic components
194 power management integrated circuit
195 energy storage device
196 sensors
198 system on a chip
199 microfluidic structure
200 microfluidic channel
202 microfluidic channel longitudinal direction
204 microfluidic channel wave shape
206 microfluidic channel semi-circular segments
210 microfluidic channel enclosure
210a lower microfluidic channel enclosure
210b upper microfluidic channel enclosure
216 inlet
220 annular rib
226 outlet
270 strain gauge sensor
274 elongated strain element
282 first end conductor
286 second end of conductor
290 leads
296 temperature sensor
300 region
θ angle

What is claimed is:

1. A wearable article, comprising:
a three-dimensionally printed base, wherein the base of the article comprises a three-dimensionally printed mesh which is formed of an elastomeric polymer composition;
a microfluidic channel coupled to the mesh, the microfluidic channel having an inlet and an outlet disposed at opposite ends of the channel, wherein the microfluidic channel is curved in a form of a serpentine shape; and wherein the serpentine shape is provided by a plurality of 180-degree semi-circular segments.

2. The article of claim 1, wherein the channel has a length from the inlet to the outlet in the range of 10-100 mm, and a channel area in a range of 0.2-0.4 mm².

3. (canceled)

4. The article of claim 1, wherein the plurality of 180-degree semi-circular segments are of a uniform diameter arranged adjacent one another between the inlet and outlet.

5. The article of claim 1, wherein a surface of the microfluid channel has a contact angle with sweat from a body when disposed thereon of less than 90 degrees.

6. The article of claim 1, wherein the microfluid channel as a cross-sectional area in a range of 0.2 to 0.4 square millimeters.

7. The article of claim 1, wherein the elastomeric polymer composition is at least one of a synthetic elastomeric polymer composition and a thermoplastic elastomeric polymer composition.

8. The article of claim 1, wherein the elastomeric polymer composition comprises at least one elastomer, wherein the at least one elastomer is a thermoplastic elastomer.

9. The article of claim 8, wherein the at least one thermoplastic elastomer comprises a thermoplastic urethane elastomer.

10. The article of claim 1, wherein the elastomeric polymer composition has at least one of the following mechanical properties:
a Shore A hardness in a range of 60-100 Shore A as measured by ASTM D2240;
a tensile strength in a range of 2-60 MPa as measured by ASTM D412;
an elongation at break in a range of 400-800% as measured by ASTM D412.

11. The article of claim 1, wherein, when disposed on a body, the mesh is flexible and stretchable as to conform to changes in a shape of the body during movement of the body.

12. The article of claim 1, further comprising:
at least partially embedding at least one electronic component in the base.

13. The article of claim 12, wherein the at least one electronic component comprises at least one sensor to collect data concerning the wearer when the article is worn by the wearer.

14. The article of claim 13, wherein the at least one sensor comprises at least one of a temperature sensor, a strain sensor, an accelerometer and a humidity sensor.

15. The article of claim 12, wherein the at least one electronic component comprises at least one antenna.

16. The article of claim 12, wherein the at least one electronic component comprises a wireless transmitter and receiver.

17. The article of claim 12, wherein the at least one electronic component 4494 comprises a data collection component.

18. The article of claim 12, wherein the at least one electronic component comprises an energy harvesting and power management component.

19. The article of claim 12, wherein the at least one electronic component comprises a wireless communication component.

20. The article of claim 1, wherein the article is a component of a system which further comprises a radio-frequency power transmitter.

21. The article of claim 1, wherein the article provides wireless communication and power charging.

22. The article of claim 1, wherein the article is battery-free.

23-41. (canceled)
